(19) United States
(12) Patent Application Publication
(10) Pub. No.: US 2024/0055129 A1
(43) Pub. Date: Feb. 15, 2024

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventors: Yasuhisa KANEKO, Kanagawa (JP); Tomohide HIRAGAMI, Kanagawa (JP); Kenji NAGAMIYA, Kanagawa (JP); Nobuya KITAMURA, Kanagawa (JP); Yasuyuki HOSONO, Kanagawa (JP)

(21) Appl. No.: 18/493,798

(22) Filed: Oct. 24, 2023

Related U.S. Application Data
(63) Continuation of application No. PCT/JP2022/019443, filed on Apr. 28, 2022.

(30) Foreign Application Priority Data
Apr. 30, 2021 (JP) 2021-077888

(51) Int. Cl. G16H 50/20

(52) U.S. Cl. CPC G16H 50/20

(57) ABSTRACT

An information processing apparatus comprising at least one processor, wherein the processor is configured to: acquire a plurality of pieces of first biological information measured with respect to a subject and assigned a date and time of measurement; acquire a plurality of pieces of second biological information that are of a different type from the first biological information and that are the second biological information measured with respect to the subject, correlated with the first biological information, and assigned a date and time of measurement; interpolate the second biological information at a time point when the first biological information is measured and when the second biological information is not measured, based on the second biological information before and after the time point; and determine a pattern corresponding to a relationship between the first biological information at the time point and the interpolated second biological information.

### CROSS REFERENCE TO RELATED APPLICATIONS

This application is a continuation of International Application No. PCT/JP2022/019443, filed on Apr. 28, 2022, which claims priority from Japanese Patent Application No. 2021-077888, filed on Apr. 30, 2021. The entire disclosure of each of the above applications is incorporated herein by reference.

### BACKGROUND

### Technical Field

The present disclosure relates to an information processing apparatus, an information processing method, and an information processing program.

### Related Art

In the related art, a technology for predicting, based on biological information at a certain time point, another biological information at the time point has been known. For example, JP2009-136446A discloses receiving an electrocardiographic signal of a specimen and estimating an ultrasound image of a time slot in which an ultrasound wave is not transmitted or received, via a regression model using the electrocardiographic signal and the ultrasound image. In addition, for example, WO2020/013230A discloses estimating a glucose level in a period other than a period in which blood glucose is actually measured, from a measured glycoalbumin concentration based on a correlation between the glycoalbumin concentration and the glucose level.

Accuracy of diagnosis based on the biological information is increased in a case where the biological information during measurement is in a state suitable for diagnosis. For example, in the case of diagnosing a subject suspected of having arrhythmia by capturing a heart image, accuracy of diagnosis is increased in a case where the heart image when the arrhythmia occurs can be obtained. However, in actuality, it is less probable that the arrhythmia occurs exactly at a timing of imaging. Thus, only the heart image in a better state than that when the arrhythmia occurs, that is, the heart image not suitable for diagnosis, may be obtained.

In addition, for example, in the case of diagnosing a subject suspected of having hypertension by capturing the heart image, accuracy of diagnosis is increased in a case where the heart image in a state of a normal blood pressure can be obtained. However, it is known that some of subjects suspected of having hypertension have an increased blood pressure particularly in a medical institution because of tension and stress (so-called white coat hypertension). In the case of such a subject, even in a case where capturing the heart image in the same state as the normal state is attempted, the heart image in a different state from the normal state is obtained because the subject is in the medical institution, and it is probable that overdiagnosis is performed.

As described above, even with the same biological information (heart image), the state suitable for diagnosis may vary depending on a type of disease to be diagnosed. Therefore, a technology for providing support for accurate diagnosis by determining in which state biological information during measurement is measured based on another biological information correlated with the biological information is desired.

### SUMMARY

The present disclosure provides an information processing apparatus, an information processing system, an information processing method, and an information processing program that can provide support for accurate diagnosis.

A first aspect of the present disclosure is an information processing apparatus comprising at least one processor, in which the processor is configured to acquire a plurality of pieces of first biological information that are the first biological information measured with respect to a subject and that are assigned a date and time of measurement of the first biological information, acquire a plurality of pieces of second biological information that are of a different type from the first biological information and that are the second biological information measured with respect to the subject, correlated with the first biological information, and assigned a date and time of measurement of the second biological information, interpolate the second biological information at a time point when the first biological information is measured and when the second biological information is not measured, based on the second biological information before and after the time point, and determine a pattern corresponding to a relationship between the first biological information at the time point and the interpolated second biological information.

A second aspect of the present disclosure is provided such that in the first aspect, the processor may be configured to determine the pattern in accordance with whether or not a ratio of match between the interpolated second biological information and the second biological information at the time point derived by comparing the first biological information at the time point with correlation data having a predetermined correlation relationship between the first biological information and the second biological information falls within an allowable range.

A third aspect of the present disclosure is provided such that in the second aspect, the processor may be configured to, in a case where the ratio of match between the second biological information at the time point derived based on the correlation data and the interpolated second biological information does not fall within the allowable range, determine the pattern in accordance with a magnitude relationship between the second biological information at the time point derived based on the correlation data and the interpolated second biological information.

A fourth aspect of the present disclosure may be provided such that in any one of the first to third aspects, the processor may be configured to linearly interpolate the second biological information at the time point based on the second biological information before and after the time point.

A fifth aspect of the present disclosure is provided such that in any one of the first to fourth aspects, the time point may be a time point when the first biological information indicates abnormality.

A sixth aspect of the present disclosure is provided such that in any one of the first to fifth aspects, the first biological information may be more frequently measured than the second biological information.

A seventh aspect of the present disclosure is provided such that in any one of the first to sixth aspects, the first biological information and the second biological information may aperiodically change depending on behavior of the subject.

An eighth aspect of the present disclosure is provided such that in any one of the first to seventh aspects, the first biological information may indicate at least one of a body temperature, a heart rate, an electrocardiogram, an electromyogram, a blood pressure, an arterial oxygen saturation, a blood glucose level, or a lipid level, and the second biological information may indicate at least one of the electrocardiogram, an electroencephalogram, a medical image captured by a medical image capturing apparatus, or a result of at least one of a hematological test, an infectious disease test, a biochemical test, or a urine test.

A ninth aspect of the present disclosure is an information processing method comprising, via a computer, acquiring a plurality of pieces of first biological information that are the first biological information measured with respect to a subject and that are assigned a date and time of measurement of the first biological information, acquiring a plurality of pieces of second biological information that are of a different type from the first biological information and that are the second biological information measured with respect to the subject, correlated with the first biological information, and assigned a date and time of measurement of the second biological information, interpolating the second biological information at a time point when the first biological information is measured and when the second biological information is not measured, based on the second biological information before and after the time point, and determining a pattern corresponding to a relationship between the first biological information at the time point and the interpolated second biological information.

A tenth aspect of the present disclosure is an information processing program causing a computer to execute a process comprising acquiring a plurality of pieces of first biological information that are the first biological information measured with respect to a subject and that are assigned a date and time of measurement of the first biological information, acquiring a plurality of pieces of second biological information that are of a different type from the first biological information and that are the second biological information measured with respect to the subject, correlated with the first biological information, and assigned a date and time of measurement of the second biological information, interpolating the second biological information at a time point when the first biological information is measured and when the second biological information is not measured, based on the second biological information before and after the time point, and determining a pattern corresponding to a relationship between the first biological information at the time point and the interpolated second biological information.

According to the aspects, the information processing apparatus, the information processing method, and the information processing program of the present disclosure can provide support for appropriate diagnosis.

### BRIEF DESCRIPTION OF THE DRAWINGS

FIG. 1 is a schematic configuration diagram of an information processing system.

FIG. 2 is an example of first biological information and second biological information.

FIG. 3 is a block diagram illustrating an example of a hardware configuration of an information processing apparatus.

FIG. 4 is a block diagram illustrating an example of a functional configuration of an information processing apparatus according to a first exemplary embodiment.

FIG. 5 is an example of time series data of the first biological information and the second biological information.

FIG. 6 is an example of correlation data between the first biological information and the second biological information.

FIG. 7 is an example of the correlation data between the first biological information and the second biological information.

FIG. 8 is an example of a screen displayed on a display.

FIG. 9 is a flowchart illustrating an example of determination processing.

FIG. 10 is a block diagram illustrating an example of a functional configuration of an information processing apparatus according to a second exemplary embodiment.

FIG. 11 is an example of a predetermined condition for each pattern.

FIG. 12 is a block diagram illustrating an example of a functional configuration of a prediction unit.

FIG. 13 is an example of the screen displayed on the display.

FIG. 14 is a flowchart illustrating an example of prediction processing.

FIG. 15 is an example of the screen displayed on the display.

### DETAILED DESCRIPTION

Hereinafter, embodiments according to the disclosed technology will be described in detail with reference to the drawings.

### First Exemplary Embodiment

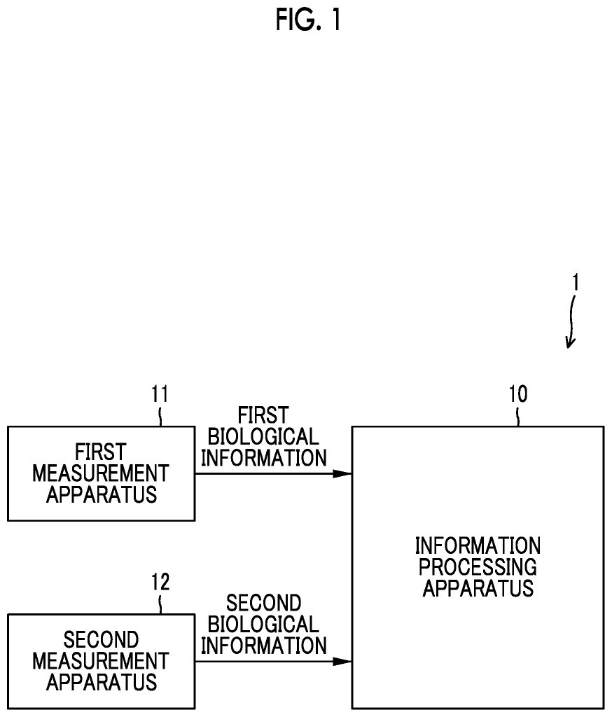

An example of a configuration of an information processing system 1 according to the present exemplary embodiment will be described with reference to FIG. 1. As illustrated in FIG. 1, the information processing system 1 comprises an information processing apparatus 10, at least one first measurement apparatus 11, and at least one second measurement apparatus 12. The information processing apparatus 10 and the first measurement apparatus 11, and the information processing apparatus 10 and the second measurement apparatus 12 can communicate with each other through wired or wireless communication.

The first measurement apparatus 11 has a function of measuring first biological information of a user over time. The first biological information may be information indicating at least one of, for example, a body temperature, a heart rate, an electrocardiogram, an electromyogram, a blood pressure, an arterial oxygen saturation (SpO2), a blood glucose level, or a lipid level. In this case, for example, a wearable terminal such as a smartwatch comprising a thermometer, a heart rate monitor, a self-monitoring blood glucose meter, and a sensor that measures biological information such as the heart rate and the arterial oxygen saturation can be applied as the first measurement apparatus 11.

The second measurement apparatus 12 has a function of measuring second biological information of the user. The second biological information is a different type of biological information from the first biological information and is less frequently measured than the first biological information (that is, the first biological information is more frequently measured than the second biological information).

The second biological information may be information indicating at least one of, for example, the electrocardiogram, an electroencephalogram, a medical image captured by a medical image capturing apparatus, or a result of at least one of a hematological test, an infectious disease test, a biochemical test, or a urine test. The medical image capturing apparatus is an apparatus that performs, for example, computed radiography (CR), computed tomography (CT), magnetic resonance imaging (MRI), ultrasound image diagnosis, fundus imaging, positron emission tomography (PET), and photoacoustic imaging (PAI). The medical image as the second biological information can be obtained using these medical image capturing apparatuses as the second measurement apparatus 12.

The hematological test is a test for obtaining, for example, a leukocyte count, an erythrocyte count, and a hemoglobin concentration as a test result. The biochemical test is a test for obtaining various indicators related to, for example, enzymes, proteins, glucose, lipids, and electrolytes as a test result. The infectious disease test is a test for obtaining presence or absence of infection caused by various infectious diseases such as, for example, influenza infection and novel coronavirus infection as a test result. The urine test is a test for obtaining, for example, glucose in urine, protein in urine, and occult blood in urine as a test result. In the case of using these various test results as the second biological information, a known analysis apparatus that analyzes, for example, blood and urine as a specimen can be applied as the second measurement apparatus 12.

Each of the first biological information and the second biological information may aperiodically change depending on behavior of a subject. The behavior of the subject includes, for example, eating, exercise, and sleeping. For example, the blood glucose level as an example of the first biological information is known to rise after the subject eats. In addition, for example, in the fundus image as an example of the second biological information, it is known that an abnormal shadow is noticeable in the state of a high blood glucose spike after eating after the subject eats.

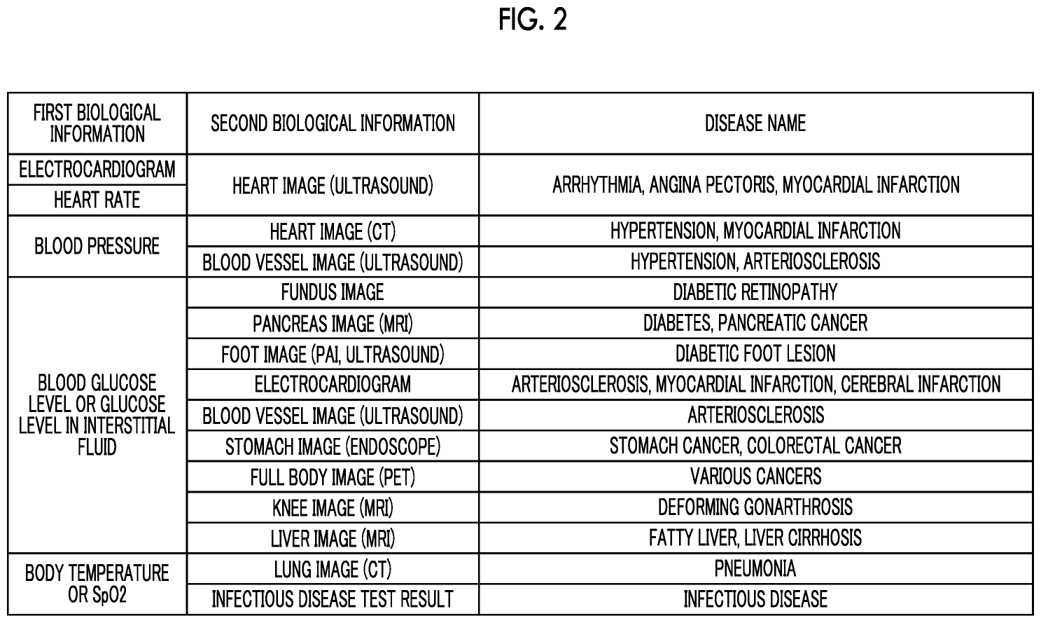

In the present exemplary embodiment, the first biological information and the second biological information are pieces of biological information that are known to be correlated with each other in advance. FIG. 2 illustrates an example of a set of the first biological information and the second biological information correlated with each other. In addition, FIG. 2 illustrates a “disease name” diagnosed based on the second biological information.

Accuracy of diagnosis based on the second biological information is increased in a case where the second biological information during measurement is in a state suitable for diagnosis. For example, in the case of diagnosing a subject suspected of having arrhythmia by capturing a heart image as the second biological information, accuracy of diagnosis is increased in a case where the heart image when the arrhythmia occurs can be obtained. However, in actuality, it is less probable that the arrhythmia occurs exactly at a timing of imaging. Thus, only the heart image in a better state than that when the arrhythmia occurs, that is, the heart image not suitable for diagnosis, may be obtained.

In addition, for example, in the case of diagnosing a subject suspected of having hypertension by capturing the heart image as the second biological information, accuracy of diagnosis is increased in a case where the heart image in a state of a normal blood pressure can be obtained. However, it is known that some of subjects suspected of having hypertension have an increased blood pressure particularly in a medical institution because of tension and stress (so-called white coat hypertension). In the case of such a subject, even in a case where capturing the heart image in the same state as the normal state is attempted, the heart image in a different state from the normal state is obtained because the subject is in the medical institution, and it is probable that overdiagnosis is performed.

As described above, even with the same second biological information, the state suitable for diagnosis may vary depending on a type of disease to be diagnosed. Therefore, the information processing apparatus 10 according to the present exemplary embodiment provides support for appropriate diagnosis by determining in which state the second biological information during measurement is measured based on the first biological information correlated with the second biological information. Hereinafter, a detailed configuration of the information processing apparatus 10 will be described.

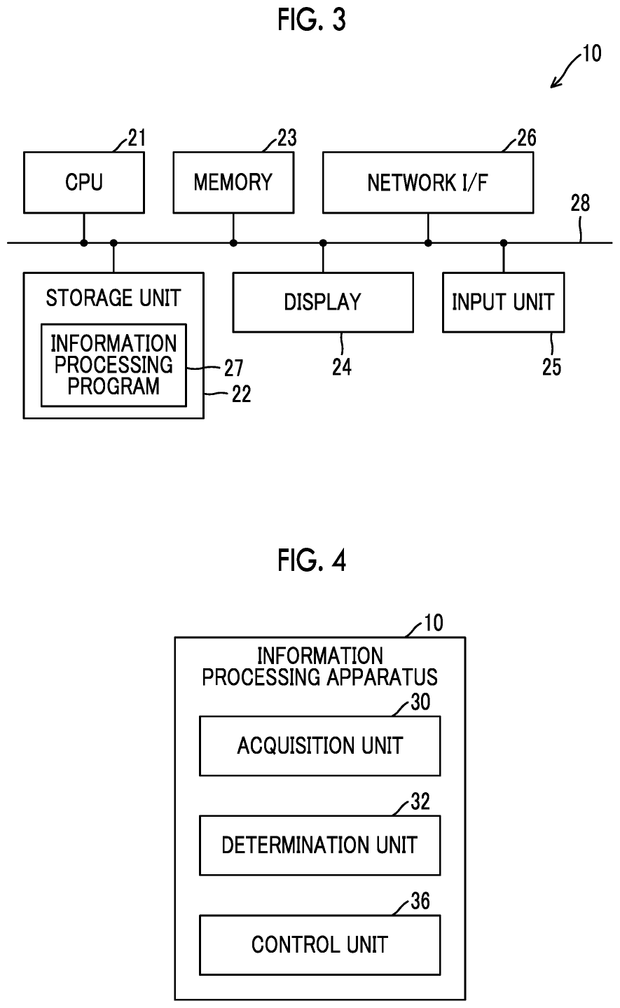

First, an example of a hardware configuration of the information processing apparatus 10 according to the present exemplary embodiment will be described with reference to FIG. 3. As illustrated in FIG. 3, the information processing apparatus 10 includes a central processing unit (CPU) 21, a non-volatile storage unit 22, and a memory 23 as a transitory storage region. In addition, the information processing apparatus 10 includes a display 24 such as a liquid crystal display, an input unit 25 such as a keyboard, a mouse, and a button, and a network interface (I/F) 26 that performs wired or wireless communication with the first measurement apparatus 11, the second measurement apparatus 12, and an external network (not illustrated). The CPU 21, the storage unit 22, the memory 23, the display 24, the input unit 25, and the network I/F 26 are connected to be capable of exchanging various information with each other through a bus 28 such as a system bus and a control bus. For example, a personal computer, a server computer, a tablet terminal, a smartphone, and a wearable terminal can be applied as the information processing apparatus 10.

The storage unit 22 is implemented by a storage medium such as, for example, a hard disk drive (HDD), a solid state drive (SSD), and a flash memory. An information processing program 27 in the information processing apparatus 10 is stored in the storage unit 22. The CPU 21 reads out and loads the information processing program 27 from the storage unit 22 into the memory 23 and executes the loaded information processing program 27. The CPU 21 is an example of a processor according to the embodiment of the present disclosure.

Next, an example of a functional configuration of the information processing apparatus 10 according to the present exemplary embodiment will be described with reference to FIG. 4. As illustrated in FIG. 4, the information processing apparatus 10 includes an acquisition unit 30, a determination unit 32, and a control unit 36. The CPU 21 functions as the acquisition unit 30, the determination unit 32, and the control unit 36 by executing the information processing program 27.

The acquisition unit 30 acquires, from the first measurement apparatus 11, a plurality of pieces of the first biological information that are the first biological information measured with respect to the subject and that are assigned a date and time of measurement of the first biological information. In addition, the acquisition unit 30 acquires, from the second measurement apparatus 12, a plurality of pieces of the second biological information that are of a different type from the first biological information and that are the second biological information measured with respect to the subject, correlated with the first biological information, and assigned a date and time of measurement of the second biological information.

In a case where the biological information acquired from the first measurement apparatus 11 and from the second measurement apparatus 12 is indicated by a numerical value (for example, the body temperature, the blood glucose level, and the leukocyte count), the acquisition unit 30 may acquire the numerical value itself as the first biological information and as the second biological information. On the other hand, in a case where the biological information acquired from the first measurement apparatus 11 and from the second measurement apparatus 12 is not indicated by a numerical value (for example, the electrocardiogram and the medical image), the acquisition unit 30 may extract a feature amount from the biological information and acquire the extracted feature amount as the first biological information and as the second biological information. For example, a known feature amount extraction technology such as a method of using a trained model that is trained in advance to take input of biological information such as the electrocardiogram and the medical image and output the feature amount can be used as a method of extracting the feature amount.

The determination unit 32 determines in which state the measured second biological information is measured based on the first biological information and the second biological information acquired by the acquisition unit 30. Specifically, the determination unit 32 determines to which of three patterns, including the state suitable for diagnosis, a better state than the state suitable for diagnosis, and a worse state than the state suitable for diagnosis, the measured second biological information corresponds. Hereinafter, a specific method of determination by the determination unit 32 will be described.

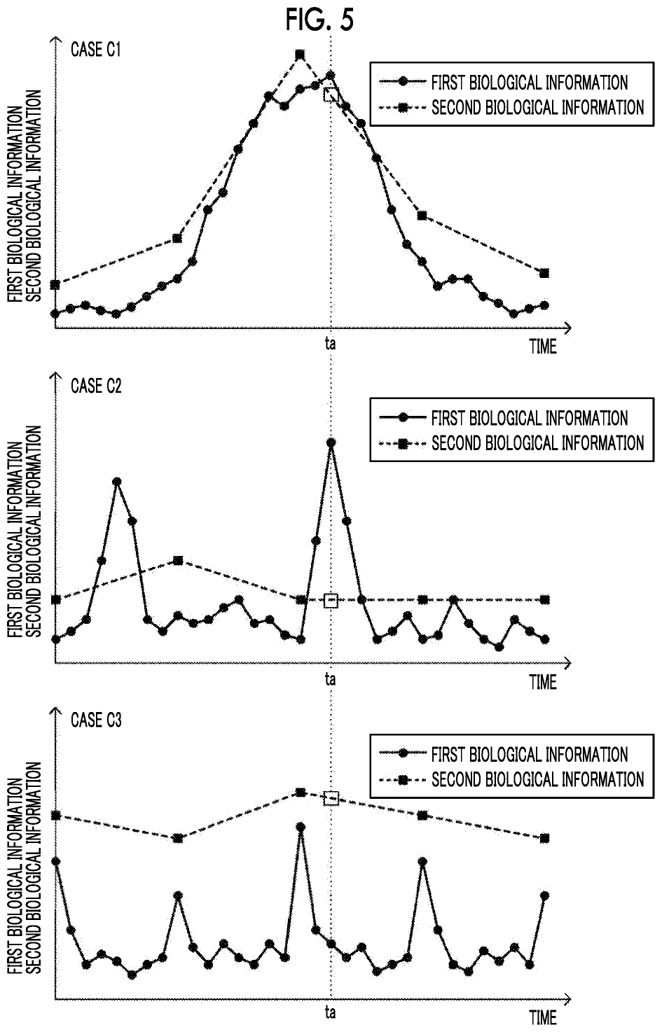

FIG. 5 illustrates an example of time series data of the first biological information and the second biological information for each of three types of cases C1 to C3 having different trends of change in the first biological information. The time series data in FIG. 5 is created based on the plurality of pieces of the first biological information and the plurality of pieces of the second biological information acquired by the acquisition unit 30 and on the dates and times of measurement assigned thereto. In FIG. 5, pieces of data that are adjacent to each other in time series are connected by a straight line. In the following description, increases in the first biological information and the second biological information indicate a worse state, and decreases in the first biological information and the second biological information indicate a better state.

The case C1 in FIG. 5 has a gentle change in the first biological information, compared to the case C2 and the case C3. The case C2 has a rapid change in the first biological information, compared to the case C1. Thus, the case C2 is a case where it is difficult to acquire the second biological information exactly when the first biological information changes, and is seen for a subject having, for example, arrhythmia. The case C3 has a rapid change in the first biological information when the second biological information is acquired, and is seen for a subject having, for example, white coat hypertension.

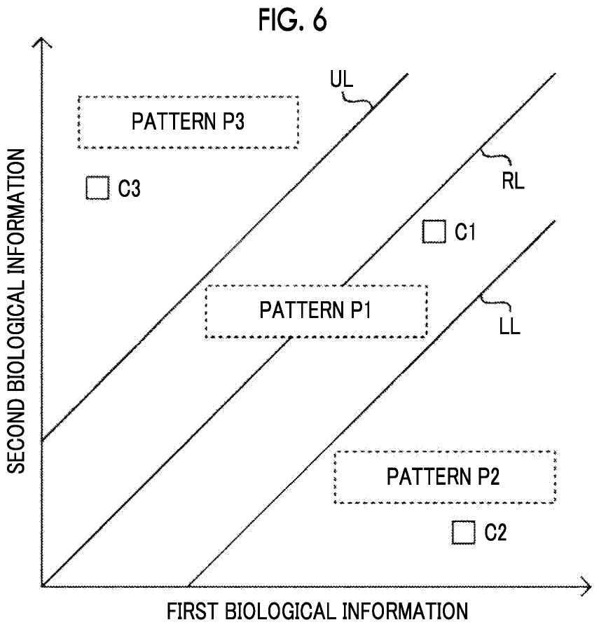

A correlation such that the second biological information is high in a case where the first biological information is high and that the second biological information is low in a case where the first biological information is low is present between the first biological information and the second biological information measured at the same time point. FIG. 6 illustrates an example of correlation data having a predetermined correlation relationship between the first biological information and the second biological information. FIG. 6 is a diagram of which a horizontal axis denotes the first biological information and a vertical axis denotes the second biological information. A regression line RL, an allowable upper limit UL, and an allowable lower limit LL are generated in advance by performing regression analysis based on records of combinations of the first biological information and the second biological information at the same time point. The regression line RL, the allowable upper limit UL, and the allowable lower limit LL in FIG. 6 are the correlation data having the predetermined correlation relationship between the first biological information and the second biological information. The correlation data is stored in advance in, for example, the storage unit 22.

The allowable upper limit UL and the allowable lower limit LL may be obtained by defining the regression line RL±σ as the allowable upper limit UL and the allowable lower limit LL, respectively, in a case where, for example, a standard deviation with respect to the regression line RL is denoted by σ. Assuming that a probability distribution of the combinations of the first biological information and the second biological information follows a normal distribution, the combinations of the first biological information and the second biological information are included between the allowable lower limit LL and the allowable upper limit UL with a probability of 34% above or below the regression line RL as a center (total 68%). That is, it is estimated that 68% of the combinations of the first biological information and the second biological information at the same time point are included between the allowable lower limit LL and the allowable upper limit UL. While the correlation data illustrated in FIG. 6 is represented by the regression line, the present disclosure is not limited thereto. The correlation data may be represented by a regression curve.

Patterns can be divided in accordance with the relationship between the first biological information and the second biological information using the regression line RL, the allowable upper limit UL, and the allowable lower limit LL. Specifically, it is assumed that a case where the combinations of the first biological information and the second biological information are between the allowable lower limit LL and the allowable upper limit UL is a pattern P1, a case where the combinations of the first biological information and the second biological information fall below the allowable lower limit LL is a pattern P2, and a case where the combinations of the first biological information and the second biological information fall above the allowable upper limit UL is a pattern P3.

In any of the cases in FIG. 5, there is a correlation between the first biological information and the second biological information measured at the same time point. Thus, it is difficult to divide the patterns based on actually measured values. Therefore, the determination unit 32 interpolates the second biological information at a time point ta when the first biological information is measured and when the second biological information is not measured, based on the second biological information before and after the time point ta (hereinafter, the interpolated second biological information at the time point ta will be referred to as an “interpolated value”). In addition, the determination unit 32 determines the patterns P1 to P3 in accordance with a relationship between the first biological information at the time point ta and the interpolated value of the interpolated second biological information at the time point ta.

The determination unit 32 may derive the interpolated value of the second biological information at the time point ta by, for example, performing linear interpolation based on the second biological information before and after the time point ta. That is, a value on a straight line connecting two pieces of the second biological information before and after the time point ta may be derived as the interpolated value of the second biological information at the time point ta. In addition, for example, the interpolated value of the second biological information at the time point ta may be derived based on an approximation curve depending on a plurality of pieces of the second biological information before and after the time point ta.

Specifically, the determination unit 32 derives the second biological information (hereinafter, referred to as a “theoretical value” of the second biological information) correlated with the first biological information at the time point ta by comparing the first biological information at the time point ta with the correlation data (regression line RL in FIG. 6). The determination unit 32 determines whether the pattern P1, the pattern P2, or the pattern P3 applies depending on whether or not a ratio of match between the theoretical value of the second biological information at the time point ta and the interpolated value of the interpolated second biological information falls within an allowable range.

A case where the ratio of match between the theoretical value of the second biological information at the time point ta and the interpolated value of the second biological information falls within the allowable range is, in other words, a case where the interpolated value of the second biological information falls between the allowable lower limit LL and the allowable upper limit UL in FIG. 6. In this case, it is determined that the pattern P1 applies. The case C1 in FIG. 5 corresponds to the pattern P1. In the case of the pattern P1, the interpolated value of the second biological information also indicates correlation with the first biological information. Thus, this means that a probability of the measured second biological information being valid is high and that a probability of the measured second biological information being in the state suitable for diagnosis is high.

On the other hand, a case where the ratio of match between the theoretical value of the second biological information at the time point ta derived based on the correlation data and the interpolated value of the interpolated second biological information does not fall within the allowable range is, in other words, a case where the interpolated value of the second biological information does not fall between the allowable lower limit LL and the allowable upper limit UL in FIG. 6. In this case, it is determined that the pattern P2 or P3 applies. In this case, the determination unit 32 may determine whether the pattern P2 or P3 applies in accordance with a magnitude relationship between the theoretical value of the second biological information at the time point ta derived based on the correlation data and the interpolated value of the interpolated second biological information.

For example, the determination unit 32 may determine that the pattern P2 applies in a case where the interpolated value of the second biological information falls below the allowable lower limit LL in FIG. 6. The case C2 in FIG. 5 corresponds to the pattern P2. In the case of the pattern P2, the interpolated value of the second biological information is lower than the theoretical value of the second biological information. Thus, this means that the actually measured value of the second biological information is probably lower than its original value, that is, the measured second biological information is probably in a better state than the state suitable for diagnosis.

In addition, for example, the determination unit 32 may determine that the pattern P3 applies in a case where the interpolated value of the second biological information falls above the allowable upper limit UL in FIG. 6. The case C3 in FIG. 5 corresponds to the pattern P3. In the case of the pattern P3, the interpolated value of the second biological information is higher than the theoretical value of the second biological information. Thus, this means that the actually measured value of the second biological information is probably higher than its original value, that is, the measured second biological information is probably in a worse state than the state suitable for diagnosis.

The time point ta used in determination can be any of time points when the first biological information is measured and when the second biological information is not measured. Among the time points, it is preferable to use a time point when the first biological information indicates abnormality as the time point ta. The time point when the first biological information indicates abnormality is, for example, a time point when the first biological information has exceeded a predetermined threshold value and a time point when the first biological information has a maximum value in a predetermined period.

On the other hand, as in the case C3 in FIG. 5, there is also a case where the first biological information indicates abnormality only when the second biological information is measured. In such a case, the determination unit 32 may use a time point when the first biological information is measured, the second biological information is not measured, and the first biological information indicates normality as the time point to used in determination.

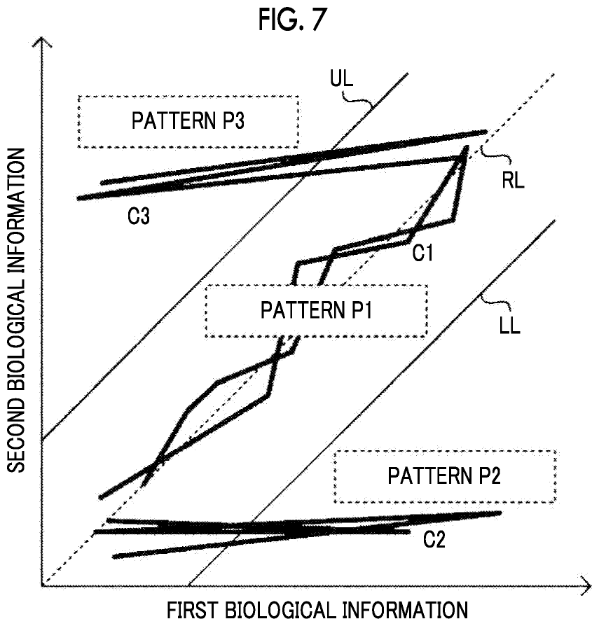

In addition, in order to improve reliability of determination, it is preferable that the determination unit 32 determines the patterns P1 to P3 in accordance with a relationship between the first biological information at a plurality of time points when the first biological information is measured and when the second biological information is not measured, and the interpolated value of the second biological information. FIG. 7 is a diagram illustrating combinations of the first biological information at the plurality of time points and the interpolated value of the second biological information in each of the cases C1 to C3 in FIG. 5 on the correlation data in FIG. 6. As illustrated in the cases C2 and C3 in FIG. 7, in a case where the combinations between the first biological information at the plurality of time points and the interpolated value of the second biological information lie between patterns, the determination unit 32 may perform determination that prioritizes the patterns P2 and P3 over the pattern P1. In addition, for example, the determination unit 32 may perform determination that prioritizes a pattern to which the combinations of the first biological information and the interpolated value of the second biological information correspond at more time points.

The control unit 36 performs a control of displaying the pattern determined by the determination unit 32 and guidance corresponding to the pattern using the display 24. For example, in a case where the determination unit 32 determines that the pattern P2 applies, the control unit 36 may provide guidance indicating that the measured second biological information is in a better state than the state suitable for diagnosis, that is, diagnosis is to be performed by considering a probability of the actual state being worse than the measured second biological information. In addition, for example, in a case where the determination unit 32 determines that the pattern P3 applies, the control unit 36 may provide guidance indicating that the measured second biological information is in a worse state than the state suitable for diagnosis, that is, diagnosis is to be performed by considering a probability of the actual state being better than the measured second biological information.

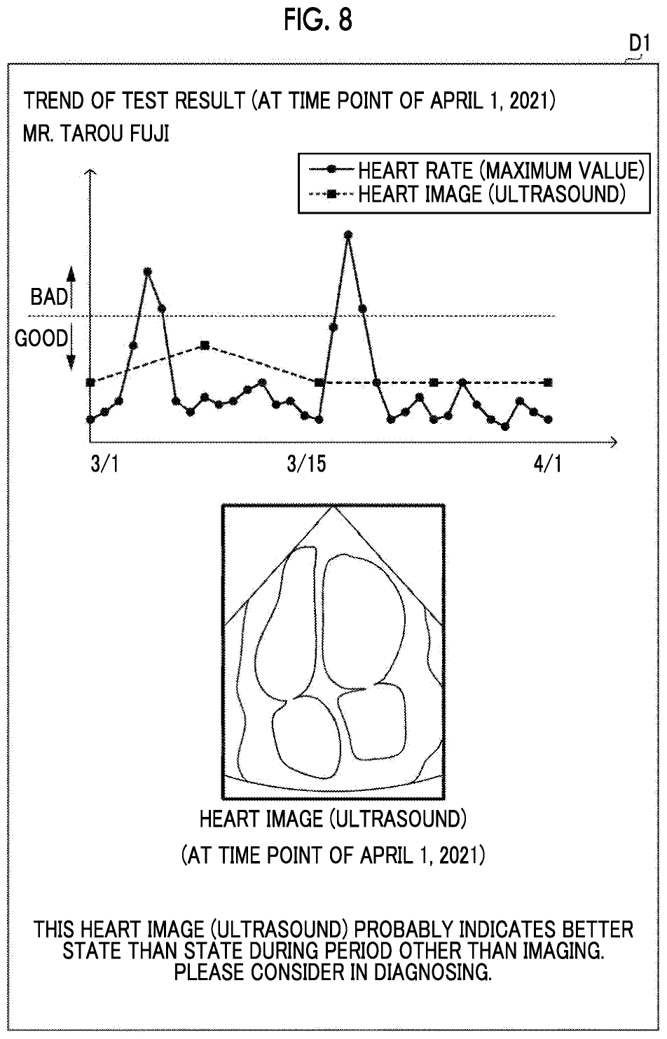

FIG. 8 illustrates an example of a screen D1 displayed on the display 24 by the control unit 36. The screen D1 in FIG. 8 corresponds to the case C2 in FIG. 5 and is a screen in a case where the determination unit 32 determines that the pattern P2 applies. In FIG. 8, the control unit 36 provides guidance indicating that diagnosis is to be performed by considering the probability of the actual state being worse than the measured second biological information.

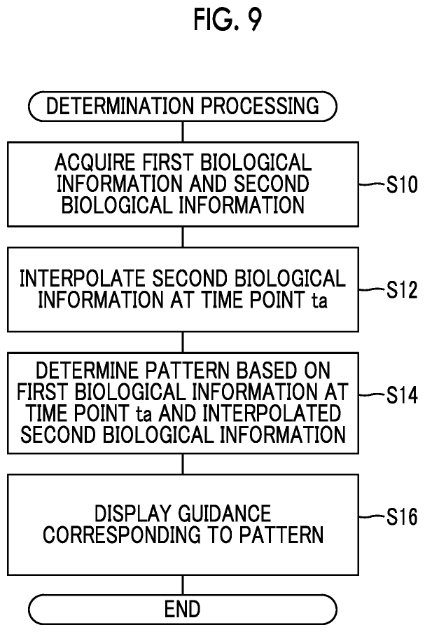

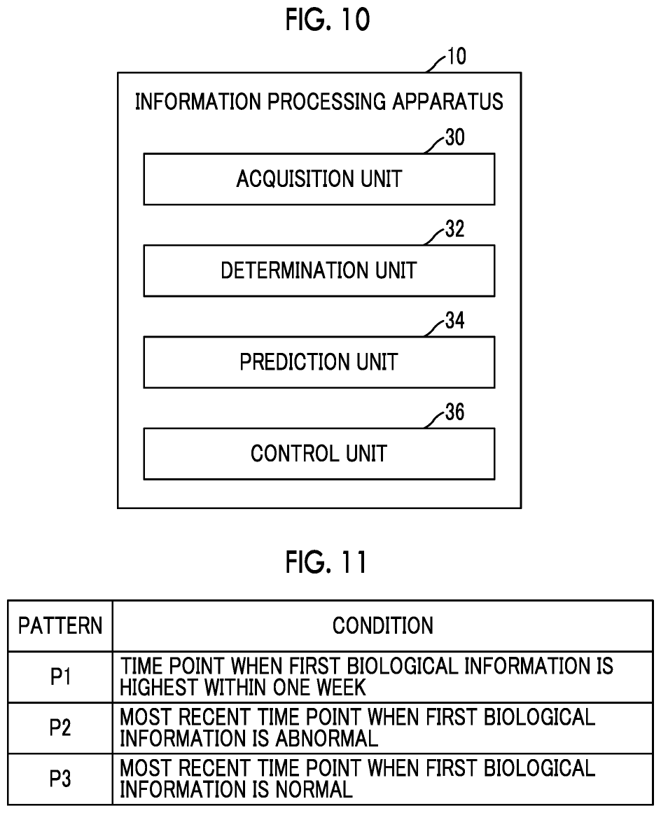

Next, action of the information processing apparatus 10 according to the present exemplary embodiment will be described with reference to FIG. 9. In the information processing apparatus 10, determination processing illustrated in FIG. 9 is executed by executing the information processing program 27 via the CPU 21. For example, the determination processing is executed in a case where an instruction to start execution is provided by the user through the input unit 25.

In step S10, the acquisition unit 30 acquires the plurality of pieces of the first biological information from the first measurement apparatus 11 and acquires the plurality of pieces of the second biological information from the second measurement apparatus 12. In step S12, the determination unit 32 interpolates the second biological information at the time point ta when the first biological information is measured and when the second biological information is not measured, based on the second biological information before and after the time point ta acquired in step S10.

In step S14, the determination unit 32 determines the patterns P1 to P3 in accordance with the relationship between the first biological information at the time point ta and the interpolated value of the second biological information at the time point ta interpolated in step S12. In step S16, the control unit 36 performs a control of displaying the guidance corresponding to the pattern determined in step S14 using the display 24 and ends the determination processing.

As described above, the information processing apparatus 10 comprises at least one processor, and the processor acquires the plurality of pieces of the first biological information that are the first biological information measured with respect to the subject and that are assigned the date and time of measurement of the first biological information, and acquires the plurality of pieces of the second biological information that are of a different type from the first biological information and that are the second biological information measured with respect to the subject, correlated with the first biological information, and assigned the date and time of measurement of the second biological information. In addition, the processor interpolates the second biological information at a time point when the first biological information is measured and when the second biological information is not measured, based on the second biological information before and after the time point and determines the pattern corresponding to the relationship between the first biological information at the time point and the interpolated second biological information. That is, according to the information processing apparatus 10, it is possible to provide support for appropriate diagnosis by determining in which state the measured second biological information is measured.

While the form of determining the pattern based on the first biological information and the second biological information of two types has been described in the first exemplary embodiment, the present disclosure is not limited thereto. For example, the acquisition unit 30 may acquire three or more types of biological information, and the determination unit 32 may determine the pattern based on the three or more types of biological information.

In addition, for example, the determination unit 32 may cause the user to select any two types of biological information among the three or more types of biological information and determine the pattern based on the selected two types of biological information. In addition, for example, the determination unit 32 may cause the user to select the type of disease to be diagnosed and determine the pattern based on predetermined two types of biological information among the three or more types of biological information for each type of disease.

### Second Exemplary Embodiment

In the first exemplary embodiment, the form of determining in which state the second biological information is measured based on the first biological information has been described. In a case where the measured second biological information is not suitable for diagnosis, it is desirable to support diagnosis by predicting and presenting the second biological information suitable for diagnosis. Therefore, the information processing apparatus 10 according to the present exemplary embodiment has a function of predicting the second biological information suitable for diagnosis in addition to the function of the first exemplary embodiment. Hereinafter, an example of a functional configuration of the information processing apparatus 10 according to the present exemplary embodiment will be described. Description of the same configuration as the first exemplary embodiment will be omitted in part.

The acquisition unit 30 acquires, from the first measurement apparatus 11, the plurality of pieces of the first biological information that are the plurality of pieces of the first biological information measured over time with respect to the subject and that are assigned the date and time of measurement of the first biological information. In addition, the acquisition unit 30 acquires, from the second measurement apparatus 12, at least one piece of the second biological information that is of a different type from the first biological information and that is measured with respect to the subject and correlated with the first biological information. The first biological information and the second biological information are the same as those in the first exemplary embodiment. Thus, description thereof will be omitted.

As described in the first exemplary embodiment, the determination unit 32 determines to which of the state suitable for diagnosis (pattern P1), a better state than the state suitable for diagnosis (pattern P2), and a worse state than the state suitable for diagnosis (pattern P3) the measured second biological information corresponds.

The prediction unit 34 receives designation of a prediction timing indicating a timing when prediction is desired with respect to the second biological information. Specifically, the prediction unit 34 designates a predetermined condition for each pattern determined by the determination unit 32 as a condition to be satisfied by the first biological information and designates a date and time when the first biological information satisfies the condition as the prediction timing. FIG. 11 illustrates an example of the predetermined condition to be satisfied by the first biological information for each of the patterns P1 to P3. Since prediction of the second biological information is performed based on the already measured first biological information (details will be described later), the prediction timing is a past time point from the current time point.

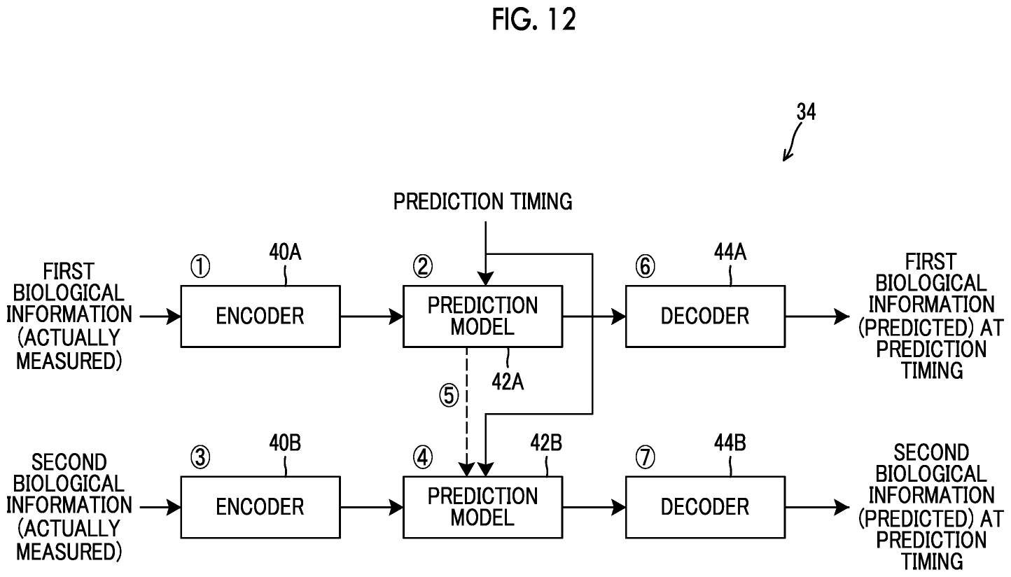

In addition, the prediction unit 34 predicts the second biological information at the designated prediction timing based on the plurality of pieces of the first biological information and the at least one piece of the second biological information acquired by the acquisition unit 30. Hereinafter, a method of predicting the second biological information will be described with reference to FIG. 12. FIG. 12 is a block diagram illustrating an example of a functional configuration of the prediction unit 34. While a circled number indicating an order of description is assigned for easy understanding in FIG. 12, this circled number does not necessarily indicate an order of processing.

First, the prediction unit 34 extracts the feature amount for each of the plurality of pieces of the first biological information acquired by the acquisition unit 30 using an encoder 40A that extracts the feature amount from the first biological information (1 in FIG. 12). For example, a trained model that is trained in advance to take input of the first biological information and output the feature amount can be applied as the encoder 40A. As such a learning model, a convolutional neural network (CNN), a residual network (ResNet), and the like may be applied, or ensemble learning that combines a plurality of learning models may be performed.

Next, the prediction unit 34 predicts the feature amount of the first biological information at the prediction timing using a prediction model 42A that generates the feature amount of the first biological information at any time point based on the feature amounts of the plurality of pieces of the first biological information (2 in FIG. 12). As the prediction model 42A, for example, it is preferable to apply a model such as a recurrent neural network (RNN) and a long short-term memory (LSTM) that can perform prediction by taking into consideration a change in time (time series data) of the first biological information indicated by the plurality of pieces of the first biological information. Particularly, applying LSTM can reflect a long-term change of the first biological information and thus, can contribute to appropriate diagnosis.

Next, the prediction unit 34 extracts the feature amount for the at least one piece of the second biological information acquired by the acquisition unit 30 using an encoder 40B that extracts the feature amount from the second biological information (3 in FIG. 12). For example, a trained model that is trained in advance to take input of the second biological information and output the feature amount can be applied as the encoder 40B. As such a learning model, CNN, ResNet, and the like may be applied, or ensemble learning that combines a plurality of learning models may be performed.

Next, the prediction unit 34 predicts the feature amount of the second biological information at the prediction timing using a prediction model 42B that generates the feature amount of the second biological information at any time point based on the feature amount of the second biological information (4 in FIG. 12). As the prediction model 42B, for example, it is preferable to apply a model such as RNN and LSTM that can perform prediction by taking into consideration a change in time (time series data) of the second biological information indicated by the plurality of pieces of the second biological information. Particularly, applying LSTM can reflect a long-term change of the second biological information and thus, can contribute to appropriate diagnosis.

Next, the prediction unit 34 corrects the feature amount of the second biological information at the prediction timing predicted by the prediction model 42B based on the feature amount of the first biological information at the prediction timing predicted by the prediction model 42A (5 in FIG. 12). That is, the prediction unit 34 predicts the second biological information by taking into consideration the change in time of the first biological information indicated by the plurality of pieces of the first biological information. Correction of the feature amount of the second biological information at the prediction timing can be performed by, for example, multimodal deep learning using the feature amount of the first biological information and the feature amount of the second biological information.

Next, the prediction unit 34 restores the first biological information at the prediction timing from the feature amount of the first biological information at the prediction timing predicted by the prediction model 42A using a decoder 44A that restores the first biological information from the feature amount of the first biological information (6 in FIG. 12). That is, according to the prediction unit 34, the first biological information at the prediction timing can also be predicted. In a case where the first biological information is acquired at the same timing as the prediction timing, the prediction unit 34 may evaluate prediction accuracy from comparison between the acquired first biological information and the predicted first biological information and present a reliability degree of prediction.

In addition, the prediction unit 34 restores the second biological information at the prediction timing from the feature amount of the second biological information at the prediction timing predicted by the prediction model 42B using a decoder 44B that restores the second biological information from the feature amount of the second biological information (7 in FIG. 12). Through the above processing, the first biological information and the second biological information at the prediction timing are predicted by the prediction unit 34.

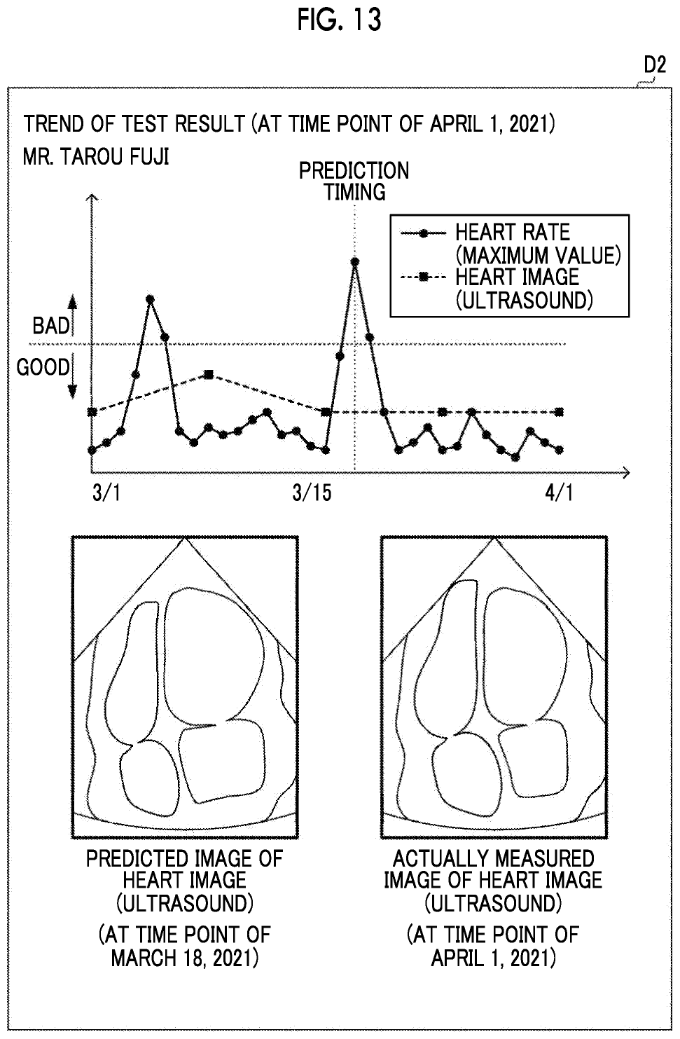

The control unit 36 performs a control of displaying the second biological information at the prediction timing predicted by the prediction unit 34 using the display 24. FIG. 13 illustrates an example of a screen D2 displayed on the display 24 by the control unit 36. In FIG. 13, the control unit 36 displays the second biological information (heart image) at the prediction timing (Mar. 18, 2021) predicted by the prediction unit 34. The control unit 36 may perform a control of displaying the first biological information at the prediction timing predicted by the prediction unit 34 and an evaluation result of the prediction accuracy based on comparison between the acquired first biological information and the predicted first biological information, using the display 24.

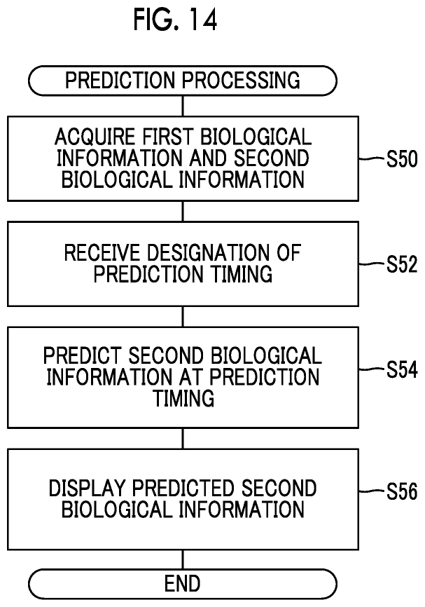

Next, action of the information processing apparatus 10 according to the present exemplary embodiment will be described with reference to FIG. 14. In the information processing apparatus 10, prediction processing illustrated in FIG. 14 is executed by executing the information processing program 27 via the CPU 21. For example, the prediction processing is executed in a case where an instruction to start execution is provided by the user through the input unit 25.

In step S50, the acquisition unit 30 acquires the plurality of pieces of the first biological information from the first measurement apparatus 11 and acquires at least one piece of the second biological information from the second measurement apparatus 12. In step S52, the prediction unit 34 receives designation of the prediction timing indicating the timing when prediction is desired with respect to the second biological information.

In step S54, the prediction unit 34 predicts the second biological information at the prediction timing received in step S52 based on the plurality of pieces of the first biological information and the at least one piece of the second biological information acquired in step S50. In step S56, the control unit 36 performs a control of displaying the second biological information at the prediction timing predicted in step S54 using the display 24 and ends the prediction processing.

As described above, the information processing apparatus 10 comprises at least one processor, and the processor acquires the plurality of pieces of the first biological information measured over time with respect to the subject, receives designation of the prediction timing indicating the timing when prediction is desired with respect to the second biological information that is of a different type from the first biological information and that is the second biological information related to the subject and correlated with the first biological information, and predicts the second biological information at the prediction timing based on the plurality of pieces of the first biological information. That is, since the second biological information at a timing suitable for diagnosis can be predicted and presented, it is possible to provide support for appropriate diagnosis.

While the form of acquiring the first biological information and the second biological information via the acquisition unit 30 and predicting the second biological information via the prediction unit 34 based on the first biological information and the second biological information has been described in the second exemplary embodiment, the present disclosure is not limited thereto. Prediction of the second biological information may be based on at least the first biological information. For example, the prediction unit 34 may predict the second biological information at the prediction timing by predicting the first biological information at the prediction timing and comparing the predicted first biological information with the correlation data between the first biological information and the second biological information. In this case, the actually measured value of the second biological information is not used in prediction of the second biological information. Thus, the acquisition unit 30 may not acquire the second biological information.

In addition, while the form of predicting the feature amount of the first biological information at the prediction timing and using the predicted feature amount of the first biological information in correcting prediction of the second biological information via the prediction unit 34 has been described in the second exemplary embodiment, the present disclosure is not limited thereto. For example, in a case where the actually measured value of the first biological information is measured at the prediction timing, prediction of the first biological information at the prediction timing may be omitted, and the actually measured value may be used in correcting prediction of the second biological information.

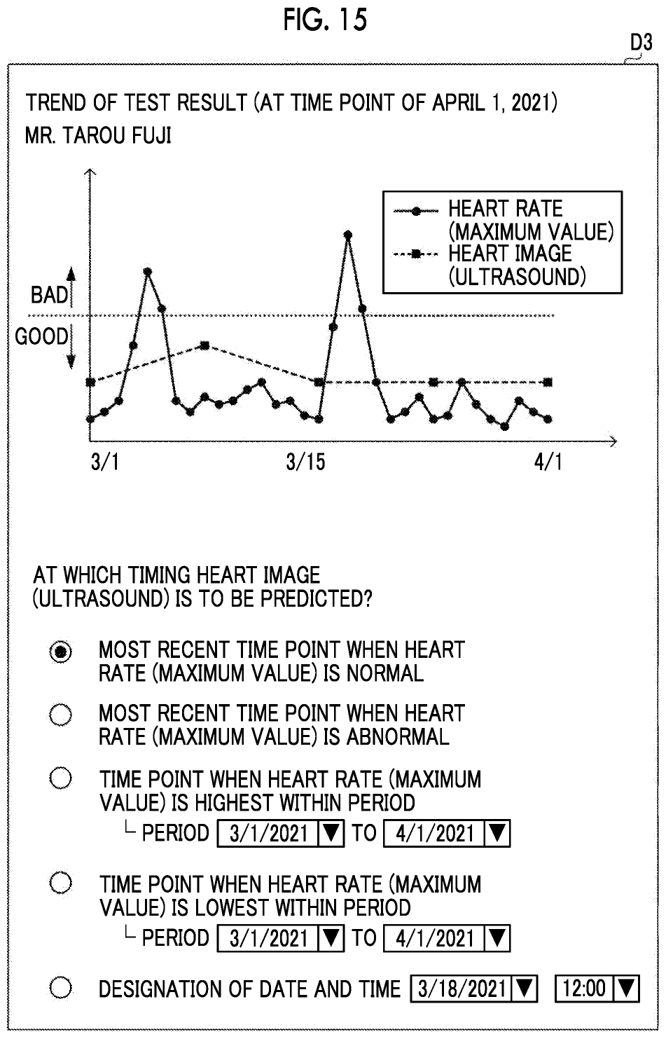

In addition, while the form of using the date and time when the first biological information satisfies the predetermined condition for each pattern determined by the determination unit 32 as the prediction timing has been described in the second exemplary embodiment, the present disclosure is not limited thereto. For example, the prediction unit 34 may receive designation of the condition to be satisfied by the first biological information from the user and designate the date and time when the first biological information satisfies the condition as the prediction timing. FIG. 15 illustrates an example of a screen D3 on which designation of the condition to be satisfied by the first biological information from the user is received. As illustrated in FIG. 15, designation of the condition to be satisfied by the first biological information may be performed by, for example, displaying the screen D3 on the display 24 via the control unit 36 and receiving designation from the user through the input unit 25. In addition, for example, as illustrated in FIG. 15, the prediction unit 34 may directly receive designation of the date and time when prediction of the second biological information is desired as the prediction timing. In these cases, the information processing apparatus 10 may not comprise the function of the determination unit 32.

In addition, the configuration of the information processing system 1 in each of the exemplary embodiments is not limited to the example illustrated in FIG. 1. For example, a part or all of the information processing apparatus 10, the first measurement apparatus 11, and the second measurement apparatus 12 included in the information processing system 1 may be an identical apparatus. In addition, for example, the information processing system 1 may comprise a plurality of the first measurement apparatuses 11 and/or a plurality of the second measurement apparatuses. In addition, each of the plurality of first measurement apparatuses 11 may measure the same type of the first biological information, or each may measure different types of the first biological information. Similarly, each of the plurality of second measurement apparatuses 12 may measure the same type of the second biological information, or each may measure different types of the second biological information.

In addition, in each of the exemplary embodiments, for example, the following various processors can be used as a hardware structure of a processing unit that executes various types of processing of the acquisition unit 30, the determination unit 32, the prediction unit 34, and the control unit 36. The various processors include, in addition to a CPU that is a general-purpose processor functioning as various processing units by executing software (program) as described above, a programmable logic device (PLD) such as a field programmable gate array (FPGA) that is a processor having a circuit configuration changeable after manufacture, a dedicated electric circuit such as an application specific integrated circuit (ASIC) that is a processor having a circuit configuration dedicatedly designed to execute specific processing, and the like.

One processing unit may be composed of one of the various processors or may be composed of a combination of two or more processors of the same type or different types (for example, a combination of a plurality of FPGAs or a combination of a CPU and an FPGA). In addition, a plurality of processing units may be composed of one processor.

A first example of a plurality of processing units composed of one processor is, as represented by computers such as a client and a server, a form of one processor composed of a combination of one or more CPUs and software, in which the processor functions as a plurality of processing units. A second example is, as represented by a system on chip (SoC) or the like, a form of using a processor that implements functions of the entire system including a plurality of processing units in one integrated circuit (IC) chip. In such a manner, various processing units are configured using one or more of the various processors as a hardware structure.

Furthermore, more specifically, an electric circuit (circuitry) in which circuit elements such as semiconductor elements are combined can be used as the hardware structure of the various processors.

While an aspect in which the information processing program 27 is stored (installed) in advance in the storage unit 22 has been described in each of the exemplary embodiments, the present disclosure is not limited thereto. The information processing program 27 may be provided in the form of a recording on a recording medium such as a compact disc read only memory (CD-ROM), a digital versatile disc read only memory (DVD-ROM), and a universal serial bus (USB) memory. In addition, the information processing program 27 may be provided in the form of a download from an external apparatus through a network. Furthermore, in addition to the information processing program, the disclosed technology is applied to a storage medium that stores the information processing program in a non-transitory manner.

In the disclosed technology, the exemplary embodiments can also be appropriately combined with each other. Above described contents and illustrated contents are detailed descriptions for parts according to the embodiment of the disclosed technology and are merely an example of the disclosed technology. For example, description related to the above configurations, functions, actions, and effects is description related to an example of configurations, functions, actions, and effects of the parts according to the embodiment of the disclosed technology. Thus, unnecessary parts may be removed, new elements may be added, or parts may be replaced in the above described contents and in the illustrated contents without departing from the gist of the disclosed technology.

The disclosure of JP2021-077888 filed on Apr. 30, 2021 is incorporated in the present specification by reference in its entirety. All documents, patent applications, and technical standards disclosed in the present specification are incorporated in the present specification by reference to the same extent as in a case where each of the documents, patent applications, and technical standards are specifically and individually indicated to be incorporated by reference.

What is claimed is:

1. An information processing apparatus comprising at least one processor, wherein the processor is configured to:
acquire a plurality of pieces of first biological information that are the first biological information measured with respect to a subject and that are assigned a date and time of measurement of the first biological information;
acquire a plurality of pieces of second biological information that are of a different type from the first biological information and that are the second biological information measured with respect to the subject, correlated with the first biological information, and assigned a date and time of measurement of the second biological information;
interpolate the second biological information at a time point when the first biological information is measured and when the second biological information is not measured, based on the second biological information before and after the time point; and
determine a pattern corresponding to a relationship between the first biological information at the time point and the interpolated second biological information.

2. The information processing apparatus according to claim 1, wherein the processor is configured to determine the pattern in accordance with whether or not a ratio of match between the interpolated second biological information and the second biological information at the time point derived by comparing the first biological information at the time point with correlation data having a predetermined correlation relationship between the first biological information and the second biological information falls within an allowable range.

3. The information processing apparatus according to claim 2, wherein the processor is configured to, in a case where the ratio of match between the second biological information at the time point derived based on the correlation data and the interpolated second biological information does not fall within the allowable range, determine the pattern in accordance with a magnitude relationship between the second biological information at the time point derived based on the correlation data and the interpolated second biological information.

4. The information processing apparatus according to claim 1, wherein the processor is configured to linearly interpolate the second biological information at the time point based on the second biological information before and after the time point.

5. The information processing apparatus according to claim 1, wherein the time point is a time point when the first biological information indicates abnormality.

6. The information processing apparatus according to claim 1, wherein the first biological information is more frequently measured than the second biological information.

7. The information processing apparatus according to claim 1, wherein the first biological information and the second biological information aperiodically change depending on behavior of the subject.

8. The information processing apparatus according to claim 1, wherein
the first biological information indicates at least one of a body temperature, a heart rate, an electrocardiogram, an electromyogram, a blood pressure, an arterial oxygen saturation, a blood glucose level, or a lipid level, and
the second biological information indicates at least one of the electrocardiogram, an electroencephalogram, a medical image captured by a medical image capturing apparatus, or a result of at least one of a hematological test, an infectious disease test, a biochemical test, or a urine test.

9. An information processing method comprising:
via a computer,
acquiring a plurality of pieces of first biological information that are the first biological information measured with respect to a subject and that are assigned a date and time of measurement of the first biological information;
acquiring a plurality of pieces of second biological information that are of a different type from the first biological information and that are the second biological information measured with respect to the subject, correlated with the first biological information, and assigned a date and time of measurement of the second biological information;
interpolating the second biological information at a time point when the first biological information is measured and when the second biological information is not measured, based on the second biological information before and after the time point; and
determining a pattern corresponding to a relationship between the first biological information at the time point and the interpolated second biological information.

10. A non-transitory computer-readable storage medium storing an information processing program causing a computer to execute a process comprising:
acquiring a plurality of pieces of first biological information that are the first biological information measured with respect to a subject and that are assigned a date and time of measurement of the first biological information;
acquiring a plurality of pieces of second biological information that are of a different type from the first biological information and that are the second biological information measured with respect to the subject, correlated with the first biological information, and assigned a date and time of measurement of the second biological information;
interpolating the second biological information at a time point when the first biological information is measured and when the second biological information is not measured, based on the second biological information before and after the time point; and
determining a pattern corresponding to a relationship between the first biological information at the time point and the interpolated second biological information.
